(19) United States
(12) Patent Application Publication
(10) Pub. No.: US 2018/0276819 A1
(43) Pub. Date: Sep. 27, 2018

(54) APPARATUS AND METHOD FOR EVALUATING QUALITY OF BINOCULAR VISION OF SUBJECT

(71) Applicants: SHENZHEN MOPTIM IMAGING TECHNIQUE CO., LTD., Shenzhen (CN); SHENZHEN CERTAINN TECHNOLOGY CO., LTD., Shenzhen (CN)

(72) Inventors: Shuguang GUO, Shenzhen (CN); Mingming WAN, Shenzhen (CN); Lei WU, Shenzhen (CN); Weihong HE, Shenzhen (CN)

(21) Appl. No.: 15/995,764

(22) Filed: Jun. 1, 2018

Related U.S. Application Data
(63) Continuation of application No. PCT/CN2016/075792, filed on Mar. 7, 2016.

(30) Foreign Application Priority Data
Dec. 30, 2015 (CN) 201511025379.2

(51) Int. Cl. G06T 7/00; G06T 7/70; A61B 3/103; A61B 3/12

(52) U.S. Cl. CPC G06T 7/0012; G06T 7/70; G06T 2207/30041; A61B 3/12; A61B 3/103

(57) ABSTRACT

An apparatus and a method for evaluating quality of binocular vision of a subject are presented. The apparatus includes a beam source generating a measurement beam which is directed to a first beam splitter; the first beam splitter for generating a first beam which is directed to a first eye, and a second beam which is directed to a second eye; a first optical micro-lens array for receiving a first return beam from the first eye to form a first plurality of light spot images; a first imaging unit for receiving the first plurality of light spot images; a second optical micro-lens array for receiving a second return beam from the second eye to form a second plurality of light spot images; a second imaging unit for receiving the second plurality of light spot images.

### CROSS-REFERENCE TO RELATED APPLICATIONS

This application is a continuation of International Application No. PCT/CN2016/075792, filed on Mar. 7, 2016, which claims priority to Chinese Patent Application No. 201511025379.2, filed on Dec. 30, 2015, the disclosures of which are hereby incorporated by reference in their entireties.

### TECHNICAL FIELD

The technology of the disclosure relates to an apparatus and a method for evaluating quality of binocular vision of a subject.

### BACKGROUND

In the prior art, although devices for measuring ocular visual acuity using geometrical optics methods can measure two eyes at the same time, usually only one parameter can be detected. For instance, in US patent (U.S. Pat. No. 5,777,718), only refractive power is measured. Chinese patent (Publication No. CN101718542B), named optical ranging device and portable refractometer thereof, provides a method and a device for measuring diopters of human eyes by measuring working distance between the human eyes and an instrument. However, only refractive power is measured.

More parameters can be measured using wavefront optics methods, but single-eye measurements are usually only possible with this method. Because wavefront measurements are sensitive to light and have high demands, it is difficult to use spectroscopic components.

International patent application (PCT Publication NO. WO2005048829A2) provides an apparatus for measuring eyesight by using a wavefront method, which can measure the two eyes simultaneously. However, it adopts complicated optical, mechanical, electrical facilities and computer software, and therefore the equipment is complicated and the cost is high. In addition, measuring accuracy of high refractive error is not high.

Chinese patent (Publication NO. CN102988022A) named ocular error detection device and method, provides an ocular error detection device and method using the wavefront optics method, but only single-eye and the refractive power of the human eye can be measured.

### BRIEF DESCRIPTION OF THE DRAWINGS

FIG. 1 illustrates a schematic diagram of an apparatus for evaluating quality of binocular vision of a subject in accordance with the first embodiment.

FIG. 2 illustrates a flow chart of a method for evaluating quality of binocular vision of a subject in accordance with the first embodiment.

FIG. 3 illustrates a diagram of a first spot pattern projected on the imaging surface of the first imaging unit and a second spot pattern projected on the imaging surface of the second imaging unit in accordance with an exemplary embodiment.

FIG. 4 illustrates a schematic diagram of an apparatus for evaluating quality of binocular vision of a subject in accordance with the second embodiment.

FIG. 5 illustrates a schematic diagram of an apparatus for evaluating quality of binocular vision of a subject in accordance with the third embodiment.

FIG. 6 illustrates a schematic diagram of an apparatus for evaluating quality of binocular vision of a subject in accordance with the fourth embodiment.

FIG. 7 illustrates a schematic diagram of an apparatus for evaluating quality of binocular vision of a subject in accordance with the fifth embodiment.

FIG. 8 illustrates a schematic diagram of a first prism and a second prism in accordance with the fifth embodiment.

FIG. 9 illustrates another schematic diagram of the first prism and the second prism in accordance with the fifth embodiment.

FIG. 10 illustrates a schematic diagram of the first prism and the second prism when the first prism and the second prism rotate outward by a degrees respectively in accordance with the fifth embodiment.

### DETAILED DESCRIPTION

The presently disclosed subject matter is described with specificity to meet statutory requirements, reference will now be made to various embodiments and specific language will be used to describe the same. It will nevertheless be understood that no limitation of the scope of the disclosure is thereby intended. Rather, the inventors have contemplated that the claimed subject might also be embodied in other ways, to include different steps or elements similar to the ones described in this document, in conjunction with present or future technologies.

Moreover, although the term “step” may be used herein to connote different aspects of methods employed, the term should not be interpreted as implying any particular order among or between various steps herein disclosed unless and except when the order of individual steps is explicitly described. And articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

### First Embodiment

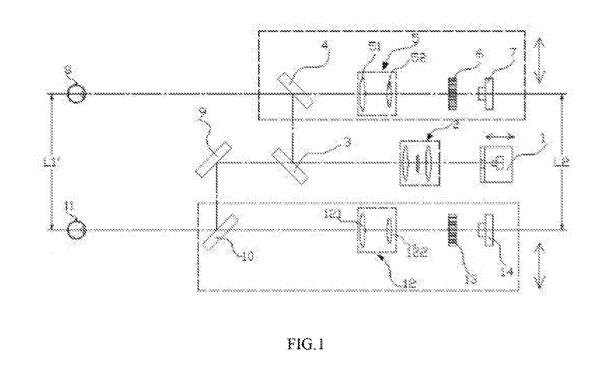

An apparatus for evaluating quality of binocular vision of a subject according to the first embodiment of the present disclosure is described. FIG. 1 illustrates a schematic diagram of an apparatus for evaluating quality of binocular vision of a subject in accordance with the first embodiment.

Referring to FIG. 1, the apparatus may include a beam source 1 for generating a measurement beam. The measurement beam may be directed to a first beam splitter 3 by a first lens set 2 which may function to collimate the measurement beam. The first beam splitter 3 may generate a first beam which is directed to a first eye 8 of the subject after passing through a second beam splitter 4, and a second beam which is directed to a second eye 11 of the subject after passing through a first reflector 9 and a third beam splitter 10.

The first lens set 2 may be formed of one single lens or a group of lenses. FIG. 1 illustrates the case where the first lens set 2 is formed of a group of lenses. In FIG. 1, the first lens set 2 includes three single lenses.

The beam source 1 is able to move along an optical axis relative to the first lens set 2. Thus, the beam source 1 is able to move to a first position where the image of the beam source 1 in the first eye 8 of the subject becomes out of focus, to fog the first eye 8 of the subject, wherein the first position is adjacent to a second position conjugated to the fundus of the first eye 8 of the subject predetermined by the first lens set 2 and optics of the first eye 8 of the subject. And the beam source 1 is able to move to a third position where the image of the beam source 1 in the second eye 11 of the subject becomes out of focus, to fog the second eye 11 of the subject, wherein the third position is adjacent to a fourth position conjugated to the fundus of the second eye 11 of the subject predetermined by the first lens set 2 and optics of the second eye 11 of the subject.

The first beam is reflected or scattered by the fundus of the first eye 8 of the subject to provide a first return beam. The first return beam is passed through the second beam splitter 4 and a second lens set 5 to a first optical micro-lens array 6, wherein the second lens set 5 may function to re-collimate the first return beam.

The second lens set 5 may be formed of one single lens or a group of lenses. FIG. 1 illustrates the case where the second lens set 5 is formed of a group of lenses. In FIG. 1, the second lens set 5 includes two single lenses 51, 52.

The first optical micro-lens array 6 functions to receive and condense the first return beam. In the structure, suppose that micro-lenses are arranged at regular intervals, and each of the micro-lenses has no aberration.

By the first optical micro-lens array 6, the first return beam is condensed. In this time, a first plurality of light spot images of the same number as the number of micro-lenses through which the first return beam has been transmitted are formed in a first condensing position. A first imaging unit 7 is disposed in the first condensing position. Each of the first plurality of light spot images is received by the first imaging unit 7. Here, in the first imaging unit 7, minute light-receiving elements are arranged in a two-dimensional manner. Therefore, it is possible to recognize positions of the respective light spot images.

The first imaging unit 7 is, for example, a charge coupled device (CCD) imaging unit or a complementary metal oxide semiconductor (CMOS) camera.

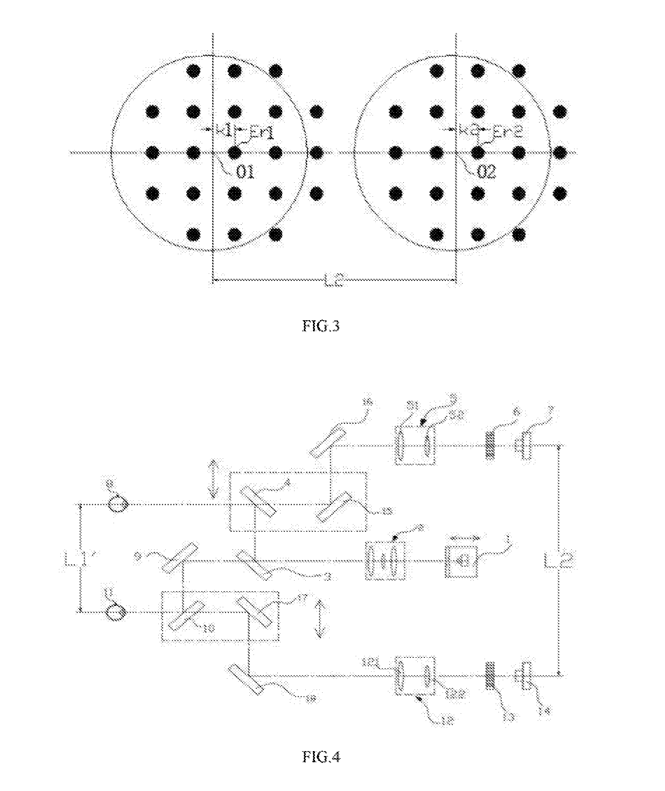

When the first return beam is incident on the first optical micro-lens array 6, the first return beam is divided by the first optical micro-lens array 6. Referring to FIG. 3, as a result, the first return beam is projected on the imaging surface of the first imaging unit 7 as a first spot pattern which includes the first plurality of light spot images. Dislocation amounts of each of the first plurality of light spot image positions from reference positions can be measured. Thus, a diopter D₁, a spherical power, an astigmatic power, and an astigmatic axis angle of the first eye may be calculated.

The second beam is reflected or scattered by the fundus of the second eye 11 of the subject to provide a second return beam. The second return beam is passed through the third beam splitter 10 and a third lens set 12 to a second optical micro-lens array 13, wherein the third lens set 12 may function to re-collimate the second return beam.

The third lens set 12 may be formed of one single lens or a group of lenses. FIG. 1 illustrates the case where the third lens set 12 is formed of a group of lenses. In FIG. 1, the third lens set 12 includes two single lenses 121, 122.

The second optical micro-lens array 13 functions to receive and condense the second return beam. In the structure, suppose that micro-lenses are arranged at regular intervals, and each of the micro-lenses has no aberration.

By the second optical micro-lens array 13, the second return beam is condensed. In this time, a second plurality of light spot images of the same number as the number of micro-lenses through which the second return beam has been transmitted are formed in a second condensing position. A second imaging unit 14 is disposed in the condensing position. Each of the second plurality of light spot images is received by the second imaging unit 14. Here, in the second imaging unit 14, minute light-receiving elements are arranged in a two-dimensional manner. Therefore, it is possible to recognize positions of the respective light spot images.

The second imaging unit 14 is, for example, a charge coupled device (CCD) imaging unit or a complementary metal oxide semiconductor (CMOS) camera.

When the second return beam is incident on the second optical micro-lens array 13, the second return beam is divided by the second optical micro-lens array 13. Referring to FIG. 3, as a result, the second return beam is projected on the imaging surface of the second imaging unit 14 as a second spot pattern which includes the second plurality of light spot images. Dislocation amounts of each of the second plurality of light spot image positions from reference positions can be measured. Thus, a diopter D₂, a spherical power, an astigmatic power, and an astigmatic axis angle of the second eye 11 of the subject may be calculated.

Prior to evaluating quality of binocular vision of the subject, the position of the second beam splitter 4, the position of the second lens set 5, the position of the first optical micro-lens array 6, the position of the first imaging unit 7, the position of the third beam splitter 10, the position of the third lens set 12, the position of the second optical micro-lens array 13, and the position of the second imaging unit 14 may be adjusted in order to direct the first beam into the first eye 8 through the pupil of the first eye 8 while the second beam is directed into the second eye 11 through the pupil of the second eye 11, and to make sure that the center Er1 of the first spot pattern coincides approximately with the center O1 of the first imaging unit 7 while the center Er2 of the second spot pattern is approximately coinciding with the center O2 of the second imaging unit 14.

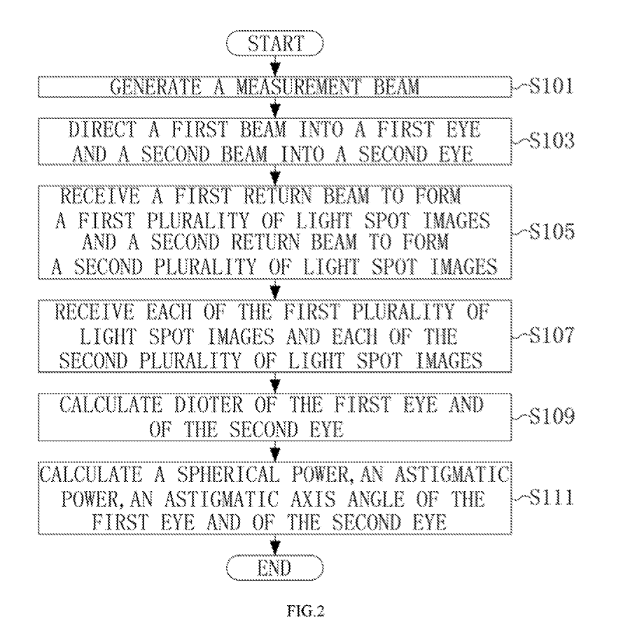

A method for evaluating quality of binocular vision of a subject according to the present embodiment is described. FIG. 2 illustrates a flow chart of a method for evaluating quality of binocular vision of a subject in accordance with the first embodiment.

Referring to FIG. 2, at Step S101, a measurement beam is generated by a beam source.

At Step S103, the measurement beam is split by a first beam splitter into a first beam which is directed into a first eye of the subject after passing through a second beam splitter and reflected or scattered by the fundus of the first eye of the subject to provide a first return beam which is passed through the second beam splitter and a second lens set to a first optical micro-lens array, and a second beam which is directed into a second eye of the subject after passing through a first reflector and a third beam splitter and reflected or scattered by the fundus of the second eye of the subject to provide a second return beam which is passed through the third beam splitter and a third lens set to a second optical micro-lens array. It is to be noted that, the measurement beam is directed to the first beam splitter by a first lens set which may function to collimate the measurement beam.

At Step S105, the first return beam is received and condensed by the first optical micro-lens array to form a first plurality of light spot images in a first condensing position, where the first plurality of light spot images forms a first spot pattern. And the second return beam is received and condensed by the second optical micro-lens array to form a second plurality of light spot images in a second condensing position, where the second plurality of light spot images forms a second spot pattern.

At Step S107, each of the first plurality of light spot images is received by a first imaging unit disposed in the first condensing position. And each of the second plurality of light spot images is received by a second imaging unit disposed in the second condensing position.

At Step S109, a diopter (D₁) of the first eye of the subject is calculated as dislocation amounts of each of the first plurality of light spot image positions from reference positions are measured. And a diopter (D₂) of the second eye of the subject is calculated as dislocation amounts of each of the second plurality of light spot image positions from reference positions are measured.

At Step S111, a spherical power, an astigmatic power, and an astigmatic axis angle of the first eye of the subject are calculated as the dislocation amounts of each of the first plurality of light spot image positions from the reference positions are measured. And a spherical power, an astigmatic power, and an astigmatic axis angle of the second eye of the subject are calculated as the dislocation amounts of each of the second plurality of light spot image positions from the reference positions are measured.

At Step S113, the pupillary distance of the subject, which is the distance between the center of the pupil of the first eye of the subject and the center of the pupil of the second eye of the subject, is measured by the method described hereinafter.

FIG. 3 illustrates a diagram of a first spot pattern projected on the imaging surface of the first imaging unit and a second spot pattern projected on the imaging surface of the second imaging unit in accordance with an exemplary embodiment. Referring to FIG. 3, K1 is the distance between the center Er1 of the first spot pattern including the first plurality of light spot images and the center O1 of the first imaging unit, K2 is the distance between the center Er2 of the second spot pattern including the second plurality of light spot images and the center O2 of the second imaging unit, and L2 is the distance between the center O1 of the first imaging unit and the center O2 of the second imaging unit.

The method for measuring pupillary distance of the subject includes: measuring the distance K1, the distance K2, the distance L2; and calculating the pupillary distance L1′ of the subject by:

$$L {1}^{′} = L 2 + \frac{K 1 ( \frac{{m}_{1} \cdot {f}_{1} \cdot {D}_{1} + 1000 {f}_{1}}{( {m}_{1} - {f}_{1} ) \cdot {D}_{1} + 1000} - \frac{{m}_{1} \cdot {f}_{1}}{{m}_{1} + {f}_{1}} )}{( \frac{{m}_{1} \cdot {f}_{1} \cdot {D}_{1} + 1000 {f}_{1}}{( {m}_{1} - {f}_{1} ) {D}_{1} + 1000} - {d}_{1} )} \cdot \frac{{m}_{1} + {f}_{1}}{{f}_{1}} + \frac{K 2 ( \frac{{m}_{2} \cdot {f}_{2} \cdot {D}_{2} + 1000 {f}_{2}}{( {m}_{2} - {f}_{2} ) \cdot {D}_{2} + 1000} - \frac{{m}_{2} \cdot {f}_{2}}{{m}_{2} + {f}_{2}} )}{( \frac{{m}_{2} \cdot {f}_{2} \cdot {D}_{2} + 1000 {f}_{2}}{( {m}_{2} - {f}_{2} ) {D}_{2} + 1000} - {d}_{2} )} \cdot \frac{{m}_{2} + {f}_{2}}{{f}_{2}} ;$$

where, d₁ is the distance between the first imaging unit and the rear principal plane of the second lens set; d₂ is the distance between the second imaging unit and the rear principal plane of the third lens set; f₁ is the focal length of the second lens set; f₂ is the focal length of the third lens set; m₁ is the distance between the pupil of the first eye 8 and the front principal plane of the second lens set; m₂ is the distance between the pupil of the second eye and the front principal plane of the third lens set.

Prior to the Step S105, the position of the beam source is adjusted to a first position where the image of the beam source in the first eye of the subject becomes out of focus, to fog the first eye of the subject, wherein the first position is adjacent to a second position conjugated to the fundus of the first eye of the subject predetermined by the first lens set and optics of the first eye of the subject, and to a third position where the image of the beam source in the second eye of the subject becomes out of focus, to fog the second eye of the subject, wherein the third position is adjacent to a fourth position conjugated to the fundus of the second eye of the subject predetermined by the first lens set and optics of the second eye of the subject. Prior to the Step S105, the position of the second beam splitter, the position of the second lens set, the position of the first optical micro-lens array, the position of the first imaging unit, the position of the third beam splitter, the position of the third lens set, the position of the second optical micro-lens array, and the position of the second imaging unit are adjusted in order to direct the first beam into the first eye through the pupil of the first eye while the second beam is directed into the second eye through the pupil of the second eye, and to make sure that the center of the first spot pattern coincides approximately with the center of the first imaging unit while the center of the second spot pattern is approximately coinciding with the center of the second imaging unit.

### Second Embodiment

An apparatus for evaluating quality of binocular vision of a subject according to the second embodiment which is a modification of the first embodiment is described. FIG. 4 illustrates a schematic diagram of an apparatus for evaluating quality of binocular vision of a subject in accordance with the second embodiment.

Referring to FIG. 4, a second reflector 15 and a third reflector 16 are disposed between the second beam splitter 4 and the second lens set 5. And a fourth reflector 17 and a fifth reflector 18 are disposed between the third beam splitter 10 and the third lens set 12.

Prior to evaluating quality of binocular vision of the subject, the position of the second beam splitter 4, the position of the second reflector 15, the position of the third beam splitter 10, and the position of the fourth reflector 17 may be adjusted in order to direct the first beam into the first eye 8 through the pupil of the first eye 8 while the second beam is directed into the second eye 11 of the subject through the pupil of the second eye 11 of the subject, and to make sure that the center Er1 of the first spot pattern coincides approximately with the center O1 of the first imaging unit 7 while the center Er2 of the second spot pattern is approximately coinciding with the center O2 of the second imaging unit 14.

### Third Embodiment

An apparatus for evaluating quality of binocular vision of a subject according to the third embodiment which is a modification of the first embodiment is described. FIG. 5 illustrates a schematic diagram of an apparatus for evaluating quality of binocular vision of a subject in accordance with the third embodiment.

Referring to FIG. 5, a first Dove prism 21 is disposed between the second beam splitter 4 and the first eye 8 of the subject. And a second Dove prism 22 is disposed between the third beam splitter 10 and the second eye 11 of the subject.

Prior to evaluating quality of binocular vision of the subject, the position of the second beam splitter 4, the position of the first Dove prism 21, the position of the third beam splitter 10, and the position of the second Dove prism 22 may be adjusted in order to direct the first beam into the first eye 8 through the pupil of the first eye 8 while the second beam is directed into the second eye 11 through the pupil of the second eye 11, and to make sure that the center Er1 of the first spot pattern coincides approximately with the center O1 of the first imaging unit 7 while the center Er2 of the second spot pattern is approximately coinciding with the center O2 of the second imaging unit 14.

### Fourth Embodiment

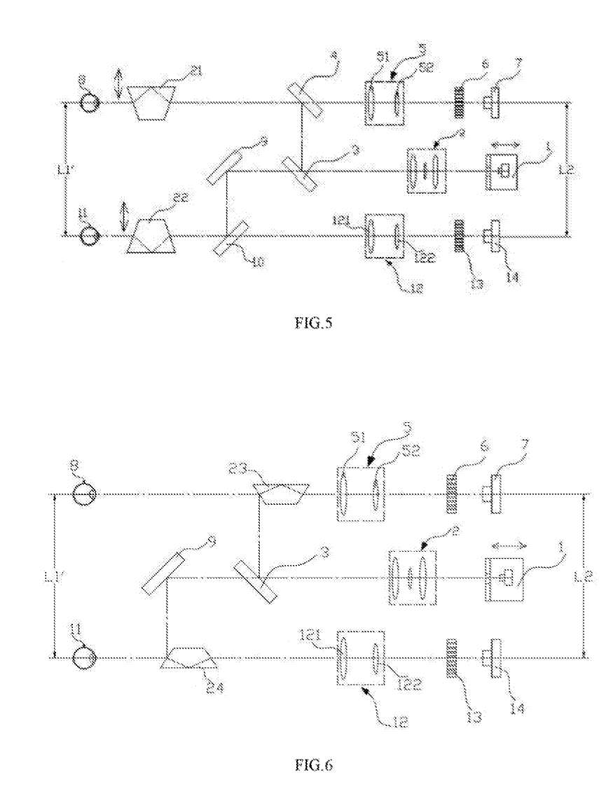

An apparatus for evaluating quality of binocular vision of a subject according to the fourth embodiment which is a modification of the first embodiment is described. FIG. 6 illustrates a schematic diagram of an apparatus for evaluating quality of binocular vision of a subject in accordance with the fourth embodiment.

Referring to FIG. 6, a third Dove prism 23 is disposed in place of the second beam splitter 4. And a fourth Dove prism 24 is disposed in place of the third beam splitter 10.

Prior to evaluating quality of binocular vision of the subject, the position of the third Dove prism 23, and the position of the fourth Dove prism 24 may be adjusted in order to direct the first beam into the first eye 8 through the pupil of the first eye 8 while the second beam is directed into the second eye 11 through the pupil of the second eye 11, and to make sure that the center Er1 of the first spot pattern coincides approximately with the center O1 of the first imaging unit 7 while the center Er2 of the second spot pattern is approximately coinciding with the center O2 of the second imaging unit 14.

### Fifth Embodiment

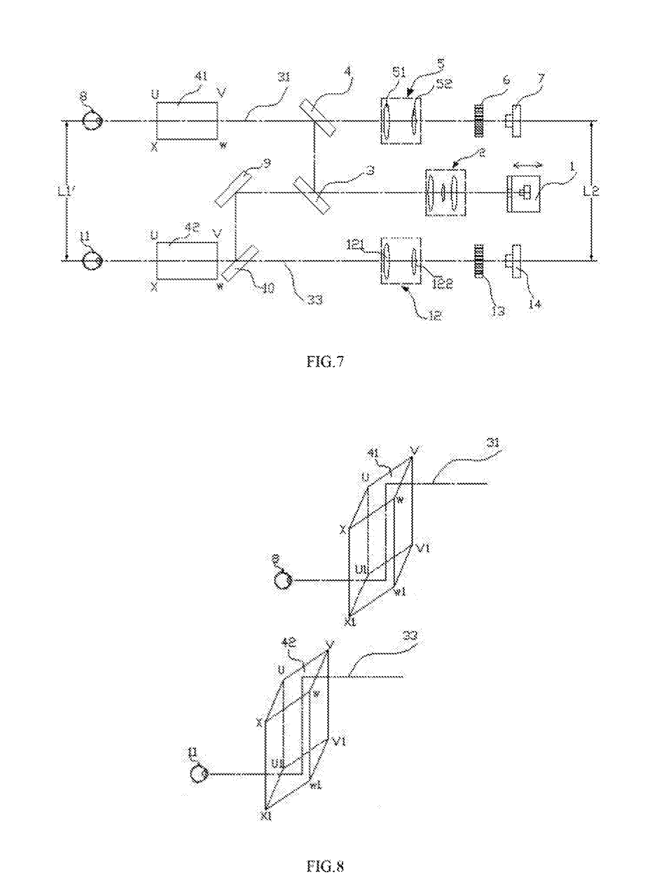

An apparatus for evaluating quality of binocular vision of a subject according to the fifth embodiment which is a modification of the first embodiment is described. FIG. 7 illustrates a schematic diagram of an apparatus for evaluating quality of binocular vision of a subject in accordance with the fifth embodiment.

Referring to FIG. 7, a first prism 41 is disposed between the second beam splitter 4 and the first eye 8 of the subject. And a second prism 42 is disposed between the third beam splitter 10 and the second eye 11 of the subject. A measurement beam generated by a beam source 1 may be directed to a first beam splitter 3 by a first lens set 2. The first beam splitter 3 may generate a first beam which is directed to a first eye 8 of the subject, after passing through a second beam splitter 4 and the first prism 41, and a second beam which is directed to a second eye 11 of the subject, after passing through a first reflector 9, a third beam splitter 10, and the second prism 42. The first beam is reflected or scattered by the fundus of the first eye 8 of the subject to provide a first return beam 31. The first return beam 31 is passed through the first prism 41, the second beam splitter 4, and a second lens set 5 to a first optical micro-lens array 6. The second beam is reflected or scattered by the fundus of the second eye 11 of the subject to provide a second return beam 33. The second return beam 33 is passed through the second prism 42, the third beam splitter 10, and a third lens set 12 to a second optical micro-lens array 13.

FIG. 8 illustrates a schematic diagram of the first prism 41 and the second prism 42 in accordance with the fifth embodiment.

Referring to FIG. 8, the first prism 41 has a slope UVWX and a slope U1V1W1X1 both of which are forming an angle of 45 degrees with a horizontal plane, while the second prism 42 has a slope UVWX and a slope U1V1W1X1 both of which are forming an angle of 45 degrees with the horizontal plane.

The first beam which is incident on the first prism 41 is reflected to the first eye 8 of the subject by the slop U1V1W1X1 after reflected by the slope UVWX to the slope U1V1W1X1. The first return beam 31 which is incident on the first prism 41 is reflected to the second beam splitter 4 by the slop UVWX after reflected by the slope U1V1W1X1 to the slope UVWX.

The second beam which is incident on the second prism 42 is reflected to the second eye 11 of the subject by the slop U1V1W1X1 after reflected by the slope UVWX to the slope U1V1W1X1. The second return beam 33 which is incident on the second prism 42 is reflected to the third beam splitter 10 by the slop UVWX after reflected by the slope U1V1W1X1 to the slope UVWX.

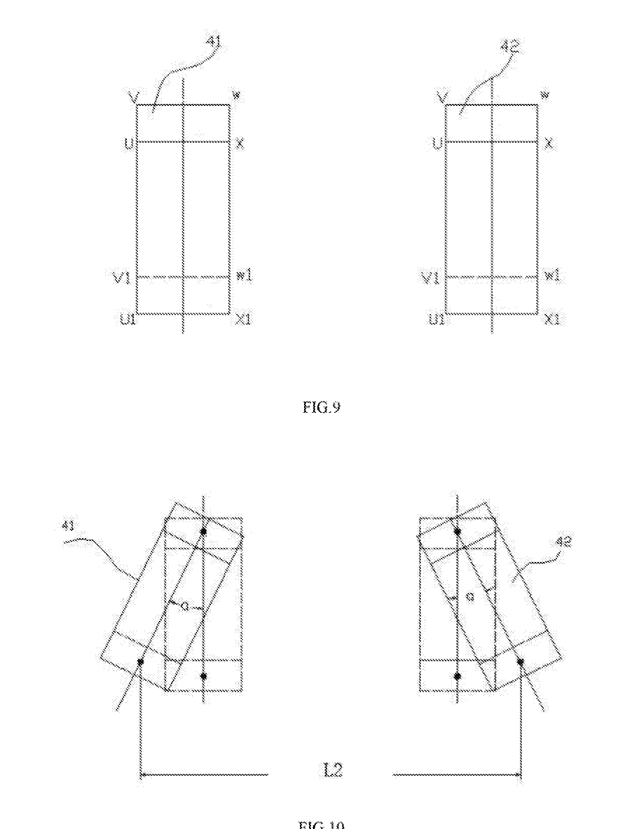

FIG. 9 illustrates another schematic diagram of the first prism 41 and the second prism 42 in accordance with the fifth embodiment. FIG. 10 illustrates a schematic diagram of the first prism 41 and the second prism 42 when the first prism 41 and the second prism 42 rotate outward by a degrees respectively in accordance with the fifth embodiment.

Referring to FIG. 9 and FIG. 10. Prior to evaluating quality of binocular vision of the subject, the first prism 41 is rotated on the plane perpendicular to an optical axis of the first return beam 31, and the second prism 42 is rotated on the plane perpendicular to an optical axis of the second return beam 33 so as to direct the first beam into the first eye 8 through the pupil of the first eye 8 while the second beam is directed into the second eye 11 through the pupil of the second eye 11, and to make sure that the center Er1 of the first spot pattern coincides approximately with the center O1 of the first imaging unit 7 while the center Er2 of the second spot pattern is approximately coinciding with the center O2 of the second imaging unit 14. FIG. 10 illuminates a case where the first prism 41 and the second prism 42 rotate outward by a degrees respectively.

While the foregoing description and drawings represent the present disclosure, it will be obvious to those skilled in the art that various changes may be made therein without departing from the true spirit and scope of the present disclosure.

What is claimed is:

1. An apparatus for evaluating quality of binocular vision of a subject, wherein the apparatus comprises:
a beam source for generating a measurement beam which is directed to a first beam splitter;
the first beam splitter for generating a first beam which is directed to a first eye of the subject and reflected or scattered by the fundus of the first eye of the subject to provide a first return beam, and a second beam which is directed to a second eye of the subject and reflected or scattered by the fundus of the second eye of the subject to provide a second return beam;
a first optical micro-lens array for receiving and condensing the first return beam to form a first plurality of light spot images in a first condensing position;
a first imaging unit disposed in the first condensing position for receiving each of the first plurality of light spot images;
a second optical micro-lens array for receiving and condensing the second return beam to form a second plurality of light spot images in a second condensing position;
a second imaging unit disposed in the second condensing position for receiving each of the second plurality of light spot images; and
a first lens set disposed between the beam source and the first beam splitter; wherein the beam source is able to:
move to a first position where the image of the beam source in the first eye of the subject becomes out of focus, to fog the first eye, wherein the first position is adjacent to a second position conjugated to the fundus of the first eye predetermined by the first lens set and optics of the first eye; and
move to a third position where the image of the beam source in the second eye of the subject becomes out of focus, to fog the second eye, wherein the third position is adjacent to a fourth position conjugated to the fundus of the second eye predetermined by the first lens set and optics of the second eye.

2. The apparatus as recited in claim 1, wherein the apparatus further comprises a second lens set optically coupled to the first optical micro-lens array, and a second beam splitter optically coupled to the second lens set, a third lens set optically coupled to the second optical micro-lens array, a third beam splitter optically coupled to the third lens set, and a first reflector coupled to direct the second beam out from the first beam splitter to the third beam splitter.

3. The apparatus as recited in claim 2, wherein the apparatus further comprises a second reflector and a third reflector that are disposed between the second beam splitter and the second lens set, and a fourth reflector and a fifth reflector that are disposed between the third beam splitter and the third lens set.

4. The apparatus as recited in claim 2, wherein the apparatus further comprises a first Dove prism disposed between the first eye and the second beam splitter, and a second Dove prism disposed between the second eye and the third beam splitter.

5. The apparatus as recited in claim 2, wherein the apparatus further comprises a first prism disposed between the first eye and the second beam splitter, and a second prism disposed between the second eye and the third beam splitter.

6. The apparatus as recited in claim 5, wherein the first prism has slopes (UVWX and U1V1W1X1) both forming an angle of 45 degrees with a horizontal plane and the second prism has slopes (UVWX and U1V1W1X1) both forming an angle of 45 degrees with the horizontal plane.

7. The apparatus as recited in claim 1, wherein
the apparatus further comprises a second lens set optically coupled to the first optical micro-lens array, a third Dove prism optically coupled to the second lens set, a third lens set optically coupled to the second optical micro-lens array, a fourth Dove prism optically coupled to the third lens set, and a first reflector coupled to direct the second beam out from the first beam splitter to the fourth Dove prism.

8. The apparatus as recited in claim 1, wherein
the first imaging unit and the second imaging unit are both complementary metal oxide semiconductor (CMOS) cameras; or
the first imaging unit and the second imaging unit are both charge coupled device (CCD) imaging units.

9. A method for evaluating quality of binocular vision of a subject, comprising:
generating a measurement beam by a beam source;
splitting, by a first beam splitter, the measurement beam into a first beam which is directed into a first eye of the subject and reflected or scattered by the fundus of the first eye of the subject to provide a first return beam, and a second beam which is directed into a second eye of the subject and reflected or scattered by the fundus of the second eye of the subject to provide a second return beam;
receiving and condensing, by a first optical micro-lens array, the first return beam to form a first plurality of light spot images in a first condensing position, wherein the first plurality of light spot images forms a first spot pattern, and receiving and condensing, by a second optical micro-lens array, the second return beam to form a second plurality of light spot images in a second condensing position, wherein the second plurality of light spot images forms a second spot pattern;
receiving each of the first plurality of light spot images by a first imaging unit disposed in the first condensing position and each of the second plurality of light spot images by a second imaging unit disposed in the second condensing position;
calculating a diopter (D₁) of the first eye of the subject after dislocation amounts of each of the first plurality of light spot image positions from reference positions are measured, and a diopter (D₂) of the second eye of the subject after dislocation amounts of each of the second plurality of light spot image positions from reference positions are measured.

10. The method as recited in claim 9, wherein the method further comprises:
splitting, by the first beam splitter, the measurement beam into the first beam which is directed into the first eye of the subject after passing through a second beam splitter and reflected or scattered by the fundus of the first eye of the subject to provide the first return beam which is passed through the second beam splitter and a second lens set to the first optical micro-lens array, and the second beam which is directed into the second eye of the subject after passing through a first reflector and a third beam splitter and reflected or scattered by the fundus of the second eye of the subject to provide the second return beam which is passed through the third beam splitter and a third lens set to the second optical micro-lens array, wherein the measurement beam is directed to the first beam splitter by a first lens set which functions to collimate the measurement beam.

11. The method as recited in claim 9, wherein
the diopter (D₁) of the first eye is calculated after adjusting the position of the beam source to a first position where the image of the beam source in the first eye becomes out of focus, to fog the first eye, wherein the first position is adjacent to a second position conjugated to the fundus of the first eye predetermined by the first lens set and optics of the first eye; and
the diopter (D₂) of the second eye is calculated after adjusting the position of the beam source to a third position where the image of the beam source in the second eye becomes out of focus, to fog the second eye, wherein the third position is adjacent to a fourth position conjugated to the fundus of the second eye predetermined by the first lens set and optics of the second eye.

12. The method as recited in claim 9, further comprising:
calculating, after the dislocation amounts of each of the first plurality of light spot image positions from reference positions are measured, a spherical power, an astigmatic power, and an astigmatic axis angle of the first eye; and
calculating, after the dislocation amounts of each of the second plurality of light spot image positions from reference positions are measured, a spherical power, an astigmatic power, and an astigmatic axis angle of the second eye.

13. The method as recited in claim 9, further comprising:
adjusting the position of the second beam splitter, the position of the second lens set, the position of the first optical micro-lens array, the position of the first imaging unit, the position of the third beam splitter, the position of the third lens set, the position of the second optical micro-lens array, and the position of the second imaging unit in order to direct the first beam into the first eye through the pupil of the first eye while the second beam is directed into the second eye through the pupil of the second eye.

14. The method as recited in claim 13, wherein the method further comprises:
measuring the distance K1 between the center (Er1) of the first spot pattern and the center (O1) of the first imaging unit;
measuring the distance K2 between the center (Er2) of the second spot pattern and the center (O2) of the second imaging unit;
measuring the distance L2 between the center (O1) of the first imaging unit and the center (O2) of the second imaging unit; and
calculating the pupillary distance L1′ of the subject by:
$$L {1}^{′} = L 2 + \frac{K 1 ( \frac{{m}_{1} \cdot {f}_{1} \cdot {D}_{1} + 1000 {f}_{1}}{( {m}_{1} - {f}_{1} ) \cdot {D}_{1} + 1000} - \frac{{m}_{1} \cdot {f}_{1}}{{m}_{1} + {f}_{1}} )}{( \frac{{m}_{1} \cdot {f}_{1} \cdot {D}_{1} + 1000 {f}_{1}}{( {m}_{1} - {f}_{1} ) {D}_{1} + 1000} - {d}_{1} )} \cdot \frac{{m}_{1} + {f}_{1}}{{f}_{1}} + \frac{K 2 ( \frac{{m}_{2} \cdot {f}_{2} \cdot {D}_{2} + 1000 {f}_{2}}{( {m}_{2} - {f}_{2} ) \cdot {D}_{2} + 1000} - \frac{{m}_{2} \cdot {f}_{2}}{{m}_{2} + {f}_{2}} )}{( \frac{{m}_{2} \cdot {f}_{2} \cdot {D}_{2} + 1000 {f}_{2}}{( {m}_{2} - {f}_{2} ) {D}_{2} + 1000} - {d}_{2} )} \cdot \frac{{m}_{2} + {f}_{2}}{{f}_{2}} ;$$
wherein, d₁ is the distance between the first imaging unit and the rear principal plane of the second lens set; d₂ is the distance between the second imaging unit and the rear principal plane of the third lens set; f₁ is the focal length of the second lens set; f₂ is the focal length of the third lens set; m₁ is the distance between the pupil of the first eye and the front principal plane of the second lens set; m₂ is the distance between the pupil of the second eye and the front principal plane of the third lens set.
